(19) United States
(12) Patent Application Publication
(10) Pub. No.: US 2008/0154331 A1
(43) Pub. Date: Jun. 26, 2008

(54) Device for multicentric brain modulation, repair and interface

(76) Inventors: Varghese John, San Francisco, CA (US); Douglas S. Kondziolka, Pittsburgh, PA (US)

Correspondence Address: GORDON & REES LLP, 101 WEST BROADWAY, SUITE 1600, SAN DIEGO, CA, 92101, US

(21) Appl. No.: 11/644,772

(22) Filed: Dec. 21, 2006

(51) Int. Cl. A61N 1/05

(52) U.S. Cl. 607/45

(57) ABSTRACT

A brain stimulation device, including: a cranial chip that is configured to be surgically implanted between a patient's scalp and skull; at least three stimulation leads connected to the cranial chip, wherein each lead has a plurality of stimulation electrodes thereon; control circuitry in the cranial chip for controlling the operation of the stimulation leads and stimulation electrodes; and a power source in the cranial chip for powering the simulation leads and the stimulation electrodes and the control circuitry.

### FIELD OF THE INVENTION

The present invention provides a system and apparatus for modulating multiple neural networks in the brain through delivering electrical pulses or receiving signals from the brain. The present invention optionally receives signals from a hand controller that would help modulate its function. The present invention is implanted under the human scalp, sitting on top of the skull and has multiple thin electrode leads reaching into different parts of the brain. Each of the leads preferably has a plurality of electrodes thereon.

### BACKGROUND OF THE INVENTION

People of all ages develop complex neurologic and behavioral disorders. These disorders are common. Such problems include Parkinson's Disease, Tremor, Depression, chronic pain and other behavioral illnesses such as obsessive compulsive disorder. The symptoms can begin at a young age and require solutions that work for decades. Standard treatments with medication help some patients but many are left with significant and life-threatening disease. For problems such as stroke, neuromodulation may improve brain functional recovery—a common health care problem not helped in any way by medication. Over the past decade, much has been learned about these disorders using basic science and functional or anatomical imaging methods.

Basic science and functional imaging have dramatically increased the knowledge of “Circuits” involved in such brain disorders. These neuronal circuits are most often multicentric and act through inhibitory and excitory feedback loops. Medication therapies for these disorders affect the brain and impact on these circuits in various ways to try and improve the disorder. However many patients are not helped adequately by medication therapies and many others grow refractory to medication therapies over time. For such patients Neuromodulation therapy can be and important treatment approach. Current brain neuromodulation devices are unifocal and can modulate one target in the brain.

Electrical stimulation of a deep brain structure has led to improvements of these disorders. Cortical brain surface stimulation may help improve stroke recovery or behavior. Optimally, patients require an efficient way to modify the aberrant function of the dysfunctional circuitry, not just a point in that system.

Complex neurological disorders act through “Circuits” with inhibitory & excitory loops. Neurocircuits involved in most disorders are multicentric and are Systems (interconnected). Many disorders of the human central nervous system are associated with abnormal patterns of physiologic activity in brain circuitry. Stimulation at one location may be inadequate for optimal patient improvement. Currently no easy, efficient or comfortable way exists to modulate multicentric brain systems simultaneously.

Debilitating movement disorders have been treated by non-reversible surgical ablation of affected brain circuits, for example by procedures such as thalamotomy or pallidotomy. Deep brain stimulation (DBS) therapy is an attractive alternative to such permanent surgeries, providing the distinct advantages of reversibility and adjustability of treatment over time. DBS is a treatment that aims to change the rates and patterns of activity of brain cells by implanting a brain stimulator (i.e., an electrode, also known as a lead) into a target region in the brain known to be associated with movement, including the thalamus, subthalamic nucleus (STN), globus pallidus, internal capsule, and nucleus accumbens.

DBS is a surgical technique first used in humans over 25 years ago. DBS has been used in a wide variety of brain targets, including the thalamus, globus pallidus and the subthalamic nucleus. Diseases that have been commonly treated with DBS include chronic pain syndromes and movement disorders including essential tremor, Parkinson's disease and dystonia. Other indications for DBS are being explored, including cluster headache, persistent vegetative state, epilepsy, and psychiatric disorders including obsessive-compulsive disorder and intractable depression.

Electrical stimulation by DBS of a particular target region of the brain, in some cases bilaterally (i.e., using an electrode on each side of the brain to stimulate i paired target regions located on each side of the brain) has been successfully used to treat symptoms of several movement disorders. For example, it has been reported in several studies that targeting of the STN is effective to alleviate symptoms of Parkinson's disease. Other areas of the brain that have been successfully targeted for this disease include the globus pallidus internus (GPi) and the ventro-lateral thalamus (ventralis intermedius or v.i.m. nucleus). Clinical results of DBS therapy for treatment of several movement disorders, including Parkinson's disease and essential tremor, have been recently reviewed in Tronnier et al., Minim. Invas. Neurosurg. 45:91-96, 2002 and in Pollack et al., Movement Disorders 17:575-583, 2002). Despite documented successes of DBS for some forms of Parkinson's disease and essential tremor (Benabid, A. L., et al., Stereotact Funct Neurosurg, 1994. 62(1-4):76-84; Benabid, A. L., et al., J Neurol, 2001. 248 Suppl 3: 11137-47), many movement disorders are unresponsive or only partially benefited by current DBS procedures. Additionally, the success of DBS procedures can diminish over time. Thalamic lesioning (Kim, M. C., et al., J Neurol Neurosurg Psychiatry, 2002. 73(4):453-5; Deuschl, G., et al., Ann Neurol, 1999. 46(1):126-8; Krauss, J. K., et al., J Neurosurg, 1994. 80(5):810-9) and thalamic DBS (Pahwa, R., et al., Mov Disord, 2002. 17(2):404-7; Samadani, U., et al., J Neurosurg, 2003. 98(4): 888-90) have both failed to consistently alleviate tremors due to structural and post-traumatic lesions affecting the cerebellothalamic and dopaminergic systems. Surgical treatment of a similar tremor associated with multiple sclerosis has also been relatively ineffective (Berk, C, et al., J Neurosurg, 2002. 97(4):815-20; Hooper, J., et al., Br J Neurosurg, 2002. 16(2):102-9; Schulder, M., et al., Stereotact Funct Neurosurg, 1999. 72(2-4): p. 196-201). Accordingly there is a need for improved therapies for conditions involving movement disorders.

Parkinson's disease (PD) is an idiopathic neurodegenerative disorder that is characterized by the presence of tremor, rigidity, akinesia or bradykinesia (slowness of movement) and postural instability. It is believed to be caused by the loss of a specific, localized population of neurons in a region of the brain called the substantia nigra. These cells normally produce dopamine, a neurotransmitter that allows brain cells to communicate with each other. These dopaminergic cells in the substantia nigra are part of an elaborate motor circuit in the brain that runs through a series of discrete brain nuclei known as the basal ganglia that control movement. It is believed that the symptoms of PD are caused by an imbalance of motor information flow through the basal ganglia.

Conventionally, a medication known as levodopa has been the mainstay of treatment for patients with Parkinson's disease. However, long-term therapy with levodopa has several well-known complications that limit the medications effectiveness and tolerability. The first of these is the development of involuntary movements known as dyskinesias. These movements can be violent at times and as or more disabling than the Parkinson's symptoms themselves. The other frequent complication is the development of “on-off” fluctuations, where patients cycle between periods of good function (the “on” period) and periods of poor function (the “off” period). These fluctuations can become very frequent, up to 7 or more cycles per day, and can cause patients to become suddenly and unpredictably “off” to the point where they cannot move.

Lesioning procedures such as pallidotomy were known to improve the motor symptoms of Parkinson's disease, presumably by disruption of the abnormal neuronal activity in the motor circuitry of the basal ganglia. The discovery that MPTP (1-methyl-4-phenyl-1,2,3,6-tetrahydropyridine) produced a Parkinsonian-like state in non-human primates allowed eletrophysiologic study of this phenomenon by numerous investigators. The discovery that high frequency stimulation could mimic the effect of lesioning led to the use of DBS for PD in humans in the early 1990's. DBS was found to improve all of the cardinal symptoms of Parkinson's disease while allowing the patient to decrease or sometimes even eliminate the amount of levodopa medication, therefore decreasing both dyskinesia and “on-off” fluctuations.

DBS is currently the surgical treatment of choice for medically refractory Parkinson's disease. Two brain targets have been found to provide clinical benefit when chronically stimulated; the subthalamic nucleus (STN) and the internal segment of the globus pallidus (GPi). In a recent prospective, double-blinded cross-over study involving 96 patients with STN DBS and 38 patients with GPi DBS, the STN group reported an improvement in the percentage of time spent during the day with good mobility and without dyskinesia from 27% to 74%. The GPi group also reported a significant improvement, from 28% to 64%.

Although the mechanism of action is not fully understood, it is believed that DBS acts to suppress the neuronal activity in the region of the brain immediately adjacent to the stimulating electrode. This hypothesis seems to be supported by the fact that lesioning a specific structure in the brain has the same clinical effect as stimulating that same structure at high (greater than 100-150 Hz) frequency. In fact, DBS has largely replaced the older lesioning procedures (such as pallidotomy and thalamotomy) that used to be the mainstay of surgical treatment for movement disorders such as Parkinson's disease. The high frequency stimulation may act to hyperpolarize immediately adjacent neurons such that they become incapable of producing normal action potentials. An alternative hypothesis is that DBS may be altering more distant structures or even fibers from far removed nerve cells that are passing through or near the area of stimulation. Whatever the mechanism of action, DBS has a distinct advantage over the older lesioning techniques because it is an adjustable therapy and does not involve destruction of the patient's brain tissue

Prior art DBS devices have several limitations that can lead to adverse effects including infection, cutaneous erosion, and lead breaking or disconnection. One study found that 27% of 66 patients with implanted DBS devices had hardware problems, similar to the results of a study where 20 (25.3%) of 79 patients who received 124 permanent DBS electrode implants had 26 hardware-related complications.

A prior art DBS device is shown in FIG. 1 and includes an electrode 100 disposed in a targeted area of the brain. The electrode is coupled to a lead 110 held in place at the top of the skull by a securement device 120. The lead 110 is coupled to a neurostimulator 130 powered by a battery 140 by means of a lead 150. The lead 150, which averages about 15 inches in length, is implanted under the scalp and traverses the length of the patient's neck to the chest (via a connected cable) where the neurostimulator 130 and battery 140 are implanted. Implantation of the DBS device is costly as it requires two implantation sites and surgeries. The lead 150 can restrict the patient's mobility and may break. Furthermore, the battery 140 must be replaced every three to five years, or even more frequently in certain patients who use more current. Additional drawbacks of the DBS device include the risk of infection and magnetic sensitivity.

The success of the routine functional neurosurgery on the subthalamic nucleus (STN) should not hide its pitfall: the possible persistence of disabling L-DOPA-induced dyskinesias, the anecdotic emergence of behavioral or cognitive disturbance, the severity of persisting axial signs. There is clearly a need to develop novel therapeutic strategies for PD patients suffering gait and postural disturbances despite optimal medical and surgical treatment. Testing of the putative efficacy of modulating structures other than STN, as the internal pallidus, the intra-laminar thalamic complex nuclei have begun; recently, there is focus on the possibility to implant the peduncolo-pontine nucleus (PPN).

The concept of using multiple sites of the brain for stimulation is being tested in the clinic albeit with great difficulty as the current devices do not enable easy use for such an application. Data on such an application was presented by Maranello et. al., 2006 (2006 meeting of the American Society for Stereotactic and Functional Neurosurgery, Boston, June 2006). They implanted, in the same session, the CM-Pf complex together with STN (in 3 Parkinson's disease patients) and PPN plus STN (n=6). Both intra-operative and post-operative neurophysiologic assessment helped recognize the functional sub-regions and optimized the implantation of the electrode. Unified Parkinson's Disease Rating Scale (UPDRS) motor scores, as well as more specific gait assessment (Tinetti & Giladi subscore) were obtained using blinded evaluations. A significant reduction in disability was achieved through the simultaneous activation of both targets. CM-Pf activation was only slightly effective on rigidity, but consistently efficacious on freezing and on tremor partially resistant to DBS-STN. Also PPN, per se, was peculiarly effective against gait instability. In addition, four weeks after steady-state reintroduction of drug therapy, PPN (and PPN+STN) provided a significant further improvement when compared to the clinical evaluation in CAPIT.

The simultaneous implantation of STN plus an unconventional target proved efficacious and flexible, supporting the on-going studies based upon STN+Pf or STN+PPN. In addition, our procedure, targeting areas which belong to different functional sub-circuits, make it possible to acquire new understanding of basal ganglia biochemistry in strict correlation with the clinical motor status. In addition, these results could turn out as useful also for different extra-pyramidal syndrome with a poor therapeutic history, as PSP and MSA.

Major depression is the most common of all psychiatric disorders (Wang, 2003↓). It ranks among the top causes of worldwide disease burden and is the leading source of disability in adults in North America under the age of 50 (World Health Organization, 2001↓). While depression can be effectively treated in the majority of patients by either medication or some form of evidence-based psychotherapy (Abosch et al., 2003↓), up to 20% of patients fail to respond to standard interventions (Fava, 2003↓; Keller et al., 1992↓). For these patients, trial-and-error combinations of multiple medications and electroconvulsive therapy are often required (Kennedy et al., 2003↓; Abosch et al., 2003↓; Sackeim et al., 2001↓). For patients who remain severely depressed despite these aggressive approaches, new strategies are needed such as DBS to modulate pathological brain circuits in depression.

Converging clinical, biochemical, neuroimaging, and postmortem evidence suggests that depression is unlikely to be a disease of a single brain region or neurotransmitter system. Rather, it is now generally viewed as a systems-level disorder affecting integrated pathways linking select cortical, subcortical, and limbic sites and their related neurotransmitter and molecular mediators (Manji et al., 2001↓; Mayberg, 1997↓; Nemeroff, 2002↓; Nestler et al., 2002↓; Vaidya et al., 2001↓). While mechanisms driving this “system dysfunction” are not yet characterized, they are likely to be multifactorial, with genetic vulnerability, developmental insults, and environmental stressors all considered important and synergistic contributors (Caspi et al., 2003↓; Heim et al., 2000↓; Kendler et al., 2001↓). Treatments for depression can be similarly viewed within this limbic-cortical system framework, where different modes of treatment modulate specific regional targets, resulting in a variety of complementary, adaptive chemical and molecular changes that re-establish a normal mood state (Vaidya et al., 2001↓; Hyman et al., 1996↓; Mayberg, 2003↓).

Functional neuroimaging studies have had a critical role in characterizing these limbic-cortical pathways (Abosch et al., 2003↓; Drevets, 1999↓; Mayberg, 1994↓; Mayberg, 2003↓). Current studies have demonstrated consistent involvement of the subgenual cingulate (Cg25) in both acute sadness and antidepressant treatment effects, suggesting a critical role for this region in modulating negative mood states (Mayberg et al., 1999↓; Seminowicz et al., 2004↓). In support of this hypothesis, a decrease in Cg25 activity is reported with clinical response to different antidepressant treatments including specific serotonin reuptake inhibitor (SSRI) antidepressant medications, electroconvulsive therapy (ECT), repetitive transcranial magnetic stimulation (rTMS), and ablative surgery (Dougherty et al., 2003↓; Goldapple et al., 2004↓; Malizia, 1997↓; Mayberg et al., 2000↓; Mottaghy et al., 2002↓; Nobler et al., 2001↓).

In addition, Cg25 connections to the brainstem, hypothalamus, and insula have been implicated in the disturbances of circadian regulation associated with depression (sleep, appetite, libido, neuroendocrine changes) (Barbas et al., 2003↓; Freedman et al., 2000↓; Jurgens et al., 1977↓; Maclean, 1990↓; Ongur et al., 1998↓). Reciprocal pathways linking Cg25 to orbitofrontal, medial prefrontal, and various parts of the anterior and posterior cingulate cortices form the neuroanatomical substrates by which primary autonomic and homeostatic processes influence various aspects of learning, memory, motivation and reward—core behaviors altered in depressed patients (Barbas et al., 2003↓; Carmichael et al., 1996↓; Haber, 2003↓; Vogt et al., 1987↓). The use of chronic stimulation to modulate Cg25 gray matter and interconnected frontal and subcortical regions could reverse the pathological metabolic activity in these circuits and produce clinical benefits in patients with treatment-resistant depression (TRD). This study reports the use of high-frequency subgenual cingulate white matter (Cg25WM) DBS in six TRD patients (Mayberg et. al., 2005).

Other neurologic conditions such as chronic pain is clearly a target for multicentric neurostimulation as multiple areas of the brain show increased rCBF. Simultaneous targeting of such sites in the brain may prove to be of greatest benefit for patients. Other disorders for multicentric stimulation include but not limited to tremor, parkinsonian tremor, dystonic tremor, monoclonic tremor, essential tremor, poststroke tremor, post-traumatic tremor, Huntington's disease, chorea, Tourette/OCD, multiple sclerosis tremor, chronic &cluster headache, psychiatric disorders and dystonia, and neurodegenrative disorders such as Alzheimer's disease.

Since movement and behavioral disorders involve complex brain circuits, stimulation at only one location may be inadequate for optimal patient improvement. For example, Parkinson's Disease patients have abnormal inhibitory or excitatory connections between brain regions such as the subthalmic nucleus, the globus pallidus, and the thalamus. Typically, only one such area is stimulated in a given patient, with the hope that effects on the other areas will be manifest. In this new system, all three areas can be targeted so that later in the clinic, the effects of uni- or multi-focal modulation can be used if needed for the benefit of the patient. Similarly, in patients with major depression, simultaneous targeting of the anterior limb of the internal capsule, as well as Brodmann area 25, may prove to be of greatest benefit for patients.

Detection and processing of physiological signals from different regions of the brain is a difficult but nevertheless an emerging field. Neural implants to study the brain using hybrid brain-machine interfaces (HBMI) is an advancing area of research. Signals such as from thought related areas of the brain may be used to trigger external or internal prosthetic devices [Mamelak et. al., 2005]. However no simple implants are available that can detect and process signals and transmit them for generating an action.

Currently no easy, efficient or comfortable way to modulate multicentric brain circuits simultaneously. There exists a critical need in the art for a cranial system that can specifically enable stimulation of multiple brain regions and address the needs of individual patients in order to provide relief or treatment for various brain disorders.

What is needed therefore is a brain stimulation device that overcomes the disadvantages of the prior art brain neuromodulation devices. What is needed is a device that requires a single implantation site and surgery. What is needed is a device where multiple leads with electrodes can be placed in different regions of the brain served by interconnected neurocircuits. As defined herein, a neurocircuit represents a pathway from one nucleus (containing a neuron cell body and its axon) to another nucleus. In existing brain stimulation systems, one nucleus is targeted in the hope of positive effects downstream. What is instead needed is a system that is able to target multiple nuclei to maximize benefits at each point in a neurocircuit. What is also needed are leads that thin so that the presence of multiple leads is not a problem. What is also needed is a device that is rechargeable using all practical energy sources as a power source. What is also needed is a cranial device that is flexible and implantable under the scalp. What is needed is a system that can be easily programmed for use by a clinician, and further affords a simple but highly advanced control interface through which the patient may easily change stimulation parameters within acceptable limits. For example, in an ideal system, voltage or current parameters (including pulse duration and frequency) could be modified by the patient, and/or the clinician. In one ideal system, the patient may be able to control the voltage within a range of 0 to 1 volt, and the pulse duration within a change of +/−90 microseconds. What is needed is a device that enables systems modulation of the brain. What is needed is a device that enables efficient, comfortable and more effective treatment of common brain disorders. What is needed is a device that can be externally adjusted to produce optimum therapeutic effects in a patient specific manner. The device of the current invention meets all of the above needs.

### SUMMARY OF INVENTION

The device for systems level brain stimulation of the present invention overcomes the disadvantages of the prior art, fulfills the needs in the prior art, and accomplishes its various purposes and functionalities by providing a system that offers the following optional features: (1) a cranial device that is easily implantable under the scalp on top of the skull, (2) a plurality of multiple thin leads, each having a plurality of electrodes thereon, with the electrodes being specifically suited for modulating muticentric neurocircuits and can perform systems modulation (e.g.: “control”) of the brain, (3) each electrode lead is individually controllable (4) each electrode lead can be placed either in a therapeutic or diagnostic mode and (5) device is rechargeable and is wirelessly linked to a hand held controller. The system described herein may advantageously be powered by a rechargeable lithium-ion layer or layers of thin film battery. The present invention includes at least 3 electrode leads. The present invention is capable of providing many years of operation. The present invention may be easily programmed for use by a clinician, and further affords a simple but highly advanced control interface through which the patient may easily change stimulation parameters within acceptable limits.

In accordance with one aspect of the invention, a small, implantable cranial device forms a key component of the system. Advantageously, the cranial device used with the system is thin enough (few millimeters) to be implanted directly under the scalp of the patient, thereby eliminating the long lead wires and tunneling procedures that have been required with existing DBS systems.

In accordance with another key aspect of the invention, the system allows at least three electrode leads attached to the cranial device, thereby eliminating the requirement for implanting multiple independent bulky implanted pulse generator (IPG's) as shown in prior art FIG. 1, per such as being done for bilateral stimulation of deep brain structures. In the present invention, electrode leads may be inserted into the brain through either a single burr hole in the skull as done with current DBS devices or through multiple mini-burr holes through the skull.

It is a feature of the invention to provide a system that incorporates where each of the electrode leads are thin in dimension and are easily inserted into the brain. Each of the leads are preferably individually controllable. As such, each lead and electrode can be programmed to provide a stimulation or receive a signal to or from the brain. This can be achieved using a switch between therapeutic and diagnostic modes. For example, in the therapeutic mode, current can be delivered for clinical benefit, whereas in the diagnostic mode, neuronal signals can be received and processed to provide information on regional cellular activity in the brain.

It is an optional feature of the invention to provide a system that incorporates a replenishable power source, e.g., a thin film rechargeable battery, as part of, or coupled to, an implanted pulse generator, whereby the power source may be replenished, as required, in order to afford a long operating life for the system.

It is a feature of the invention that the recharging unit could be any practical energy source including but not limited to RF energy powering such as an external transmitting device, a near infrared (NIR) light transmitter (such as described by Gotto et. al., 2001), a device that converts body heat to an electrical charge, vibration energy or ultrasound energy.

It is another feature of the invention, in accordance with one embodiment thereof, to provide a cranial device system that is capable of delivering stimulation pulses to the brain through selected electrodes on at least three electrode leads connected to a single, multichannel pulse generator, whereby, unilateral, bilateral or multicentric stimulation of the brain may be provided, as desired.

There has been outlined, rather broadly, the more important features of the invention in order that the detailed description thereof that follows may be better understood, and in order that the present contribution to the art may be better appreciated. There are, of course, additional features of the invention that will be described below and which will form the subject matter of the claims appended herein.

In this respect, before explaining at least one embodiment of the invention in detail, it is to be understood that the invention is not limited in its application to the details of design and to the arrangement of components set forth in the following description or illustrated in the drawings. The invention is capable of other embodiments and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other methods and systems for carrying out the several purposes of the present invention. It is important, therefore, that the claims be regarded as including such equivalent methods and systems insofar as they do not depart from the spirit and scope of the present invention.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 illustrates a prior art Deep Brain Stimulation (DBS) device;

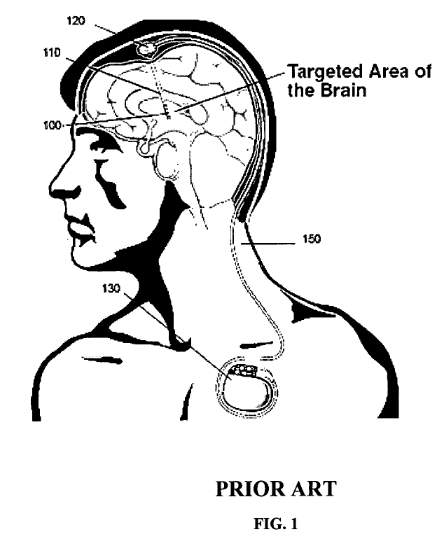

FIG. 2 illustrates the various components of the cranial device system made in accordance with the present invention;

FIG. 3 is a schematic representation of the implantation and location of the cranial device in accordance with the invention;

FIG. 4 is a schematic representation of the various elements within each of the main sub-systems of the cranial device, which sub-systems include an implantable cranial chip device (CD), a Hand-held programmer (HHP), a Physicians Programming System (PPS), a Servicing and Diagnostic System (SDS), and a Recharging System (RCS) in accordance with the invention;

FIG. 5 is a block diagram of the cranial device (CD) in accordance with the invention;

FIG. 6 is a schematic representation of the cranial device located in the patient with the electrodes in different brain regions and the HHP for modulation of the cranial device in accordance with the invention;

FIG. 7 is a schematic representation of the implantation and location of the cranial device in accordance with one aspect of the invention.

FIG. 8 is schematic representation of pair of the present devices, with each operating on different sides of the patient's brain in one aspect of the invention.

### DETAILED DESCRIPTION OF THE INVENTION

The present invention addresses the above and other needs by providing an implantable cranial device that: (1) is easily implantable in an efficient and comfortable manner, (2) is optionally rechargeable, (3) has at least three leads each with multiple electrodes specifically suited for therapeutic neuromodulation or diagnostic neurosensing, (4) can target many disease-relevant brain regions, and (5) provides for patient specific therapeutic neuromodulation. In one exemplary aspect, the present invention provides brain stimulation device, comprising: a cranial chip that is configured to be surgically implanted between a patient's scalp and skull; at least three stimulation leads connected to the cranial chip, wherein each lead has a plurality of stimulation electrodes thereon; control circuitry in the cranial chip for controlling the operation of the stimulation leads and stimulation electrodes; and a power source in the cranial chip for powering the simulation leads and the stimulation electrodes and the control circuitry.

The system described herein is optionally powered by a rechargeable lithium-ion battery. The system is capable of providing many years of operation. The system may be easily programmed for use by a clinician, and further affords a simple but highly advanced control interface through which the patient may easily change stimulation parameters within acceptable limits.

In accordance with one aspect of the invention, a pulse generator (IPG) circuit forms a key component of the cranial chip system. Advantageously, the cranial chip system is small enough to be implanted directly under the scalp of the patient, thereby eliminating the long extension cables and tunneling procedures that have been required with existing DBS systems.

In accordance with another key aspect of the invention, the cranial system allows up to three electrode leads to be attached to the cranial device, thereby eliminating the requirement for implanting multiple independent IPG's such as is currently being done currently for bilateral stimulation of deep brain structures.

It is a feature of the invention to provide a cranial system that incorporates a replenishable power source, e.g., a rechargeable battery, as part of, or coupled to, cranial device, whereby the power source may be replenished, as required, in order to afford a long operating life for the cranial system.

It is another feature of the invention to provide a recharging system. The recharging system could be any of the following but not limited to just them: an inductive source of electromagnetic energy, a light source such as a photo diode, a body heat converting source, a source that generates vibrational energy, a source that generates ultrasound.

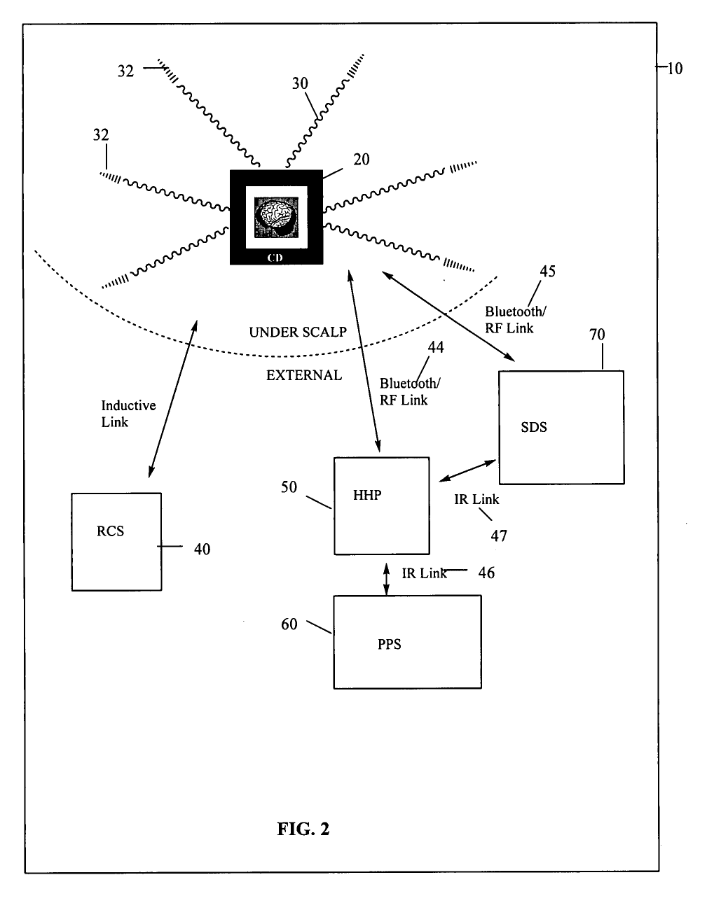

The cranial device system 10 preferably includes four major functional blocks, as seen in FIG. 2: the cranial chip device (CD) 20; The Hand-Held Programmer (HHP) 50; The Re-Charging System (RCS) 40; and the Physicians Programming System (PPS) 60. In various embodiments, the CD 20 contains a 16 bit microprocessor 21, memory 23 and 24, a rechargeable battery 27 and custom pulse generation circuitry 25 and 26. Communication to the chip 20 is via RF link 44 or other links 42 or 45. The HHP 50 takes the form of a small pager-like or PDA device, with an LCD graphics display and a direct user interface and keyboard. Preferably, the HHP 50 is able to communicate with the cranial device 20 over a comfortable distance, e.g., up to 2 feet away, allowing the patient and clinician alike simple and efficient control of the IPG. The CPS 60 may be used by the clinician to fit the cranial device 20 and electrodes 32 to the patient, and to record and document all stimulation settings. The PPS 60 preferably communicates to the HHP 50 using an InfraRed or Bluetooth type wireless link 46, a standard in the computer industry. The HHP 50 communicates to the CD 20 over an RF link such as IR or Bluetooth 44.

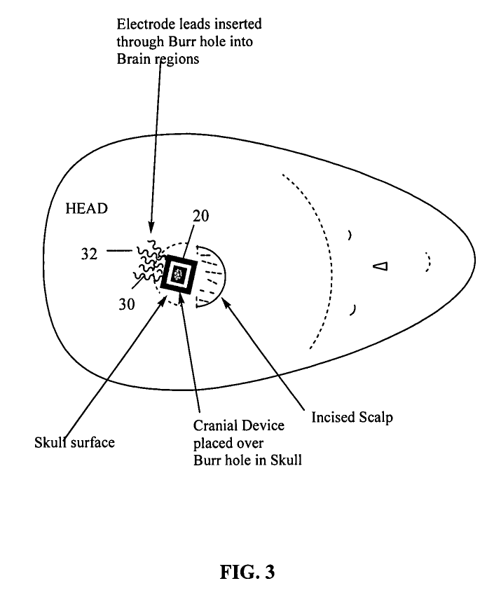

FIG. 3 shows the surgical placement of the present invention, in which the patient's scalp is incised and cranial chip 20 is placed between the patient's scalp and skull. Leads 30 then extend to various operative locations within the patient's brain. Electrodes 32 are disposed on leads 30 (positioned either at the ends or along the length of leads 30).

The following description is of the best mode presently contemplated for carrying out the invention. This description is not to be taken in a limiting sense, but is made merely for the purpose of describing the general principles of the invention. The scope of the invention should be determined with reference to the claims. The cranial device system of the present invention includes a cranium mountable pulse generator, support for at least three electrode leads for supporting stimulation of neural networks such as the brain, electrodes specifically designed for the small structures required for the brain stimulation application, an electrode positioning system such as functional imaging or anatomical imaging, and a electrode fixation system guaranteeing reliable electrode and lead wire position once implanted.

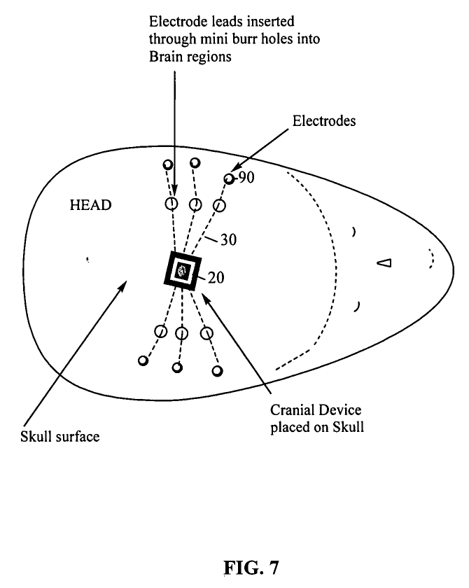

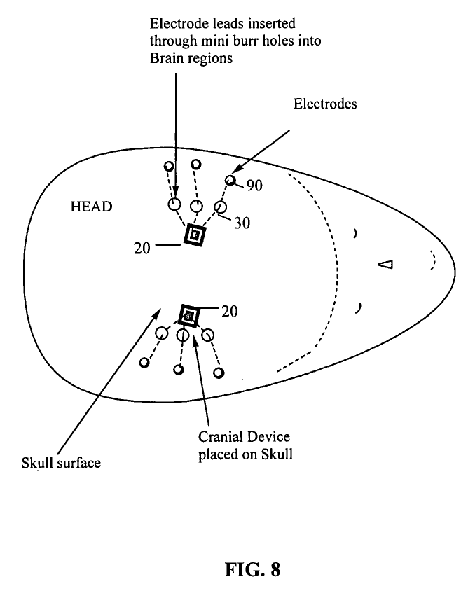

The system includes an implantable computer chip or application specific integrated circuit (ASIC). The ASIC would have the circuitry for the pulse generator (IPG), the analog and digital integrated circuit (IC), along with the ability to process & store signals and is adapted to be implanted directly under the scalp of a patient. The cranial device (CD) 20 has at least three hair-thin leads 30, each having a plurality of electrodes 32 thereon, is attached to the cranial device 20 via a suitable connector 22. Each lead includes at least two electrodes 32. In various embodiments, electrodes 32 are positioned along the length of leads 30. It is to be understood that leads 32 may also be placed at the ends of leads 30. (Note: similar electrodes 90 are shown in FIGS. 7 and 8). The cranial device 20 optionally includes a rechargeable battery. The battery is recharged, as required, from an Re-Charging system (RCS) 40, typically through an inductive link 42 or other approaches shown above.

The cranial chip device, as explained more fully below, includes a processor and other electronic circuitry that allows it to generate stimulus pulses that are applied to the patient through the electrodes 32 in accordance with a stored program. The cranial device 20 is programmed and tested through a hand held programmer (HHP) 50; a physicians programming system (PPS) 60 that uses an HHP, or equivalent, to relay information; or a servicing and diagnostic system (SDS) 70. The HHP 50 may be coupled to the cranial device 20 via an RF link 44. Similarly, the MDS 70 may be coupled to the cranial device 20 via another RF link 45. The CPS 60, which is coupled to the cranial device 20 by way of the HHP 50, may also be coupled to the HHP 50 via an infra-red or a RF Bluetooth link 46. Likewise, the MDS 70 may be coupled to the HHP via another infra-red or RF Bluetooth link 47. Other types of telecommunicative links, other than RF or infra-red may also be used for this purpose. Through these links, the PPS 60, for example, may be coupled through the HHP 50 to the cranial device 20 for programming or diagnostic purposes. The MDS may also be coupled to the cranial device 20, either directly through the RF link 45, or indirectly through the IR link 47 with the HHP 50.

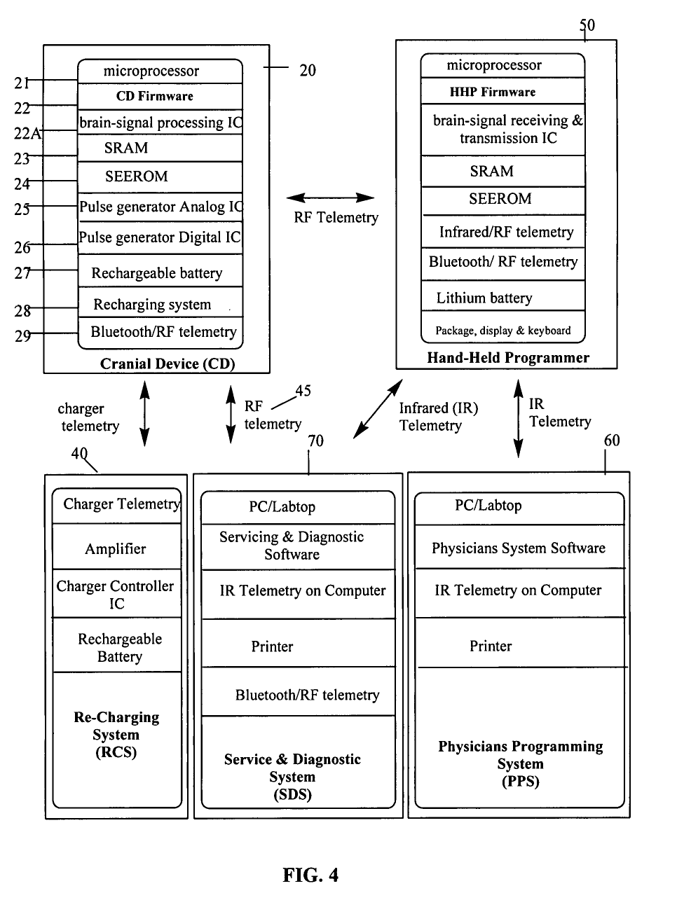

The subsystems of cranial device 20 are shown in FIG. 4, and may include various elements, including a microprocessor 21, cranial device (CD) firmware 22, a SRAM memory 23 (which SRAM memory is optional, and may not be needed in some embodiments), a SEEROM memory 24, an analog pulse generator integrated circuit (IC) 25 (which analog pulse generator circuit 25 functions as the output circuit of the IPG), a digital pulse generator IC 26, a thin film rechargeable battery 27, a battery charging system and telemetry circuit 28, and an RF telemetry circuit 29. The microprocessor 21, in the preferred embodiment, comprises a 16 bit microprocessor and associated external controller based upon the VAutomation 8086 processor, or equivalent. Advantageously, this processor 21 is a flexible 16 bit processor that has been around for years and was the processor used in the IBM PC, thus many development tools are available for both software and hardware design for this device. The general performance-based features for the core and the additional peripheral devices in the microprocessor IC 21 are summarized as follows: 1. Core: Equivalent to Intel 8086 from Vautomation, or equivalent.

Exemplary features of processor 21 may include, but are not limited to: Operating Voltage: 2.2-3.5V 3. Oscillator-1. 048 MHz crystal controlled oscillator, under 1 uA current consumption, 2.2-3.5V supply 4. Address Bus: 20 bit, non-multiplexed 5. Data Bus: 16 bit, non-multiplexed, supports multiplexed with CPUALE signal 6. Power Consumption: 300 uA @ 1 MHz main crystal frequency 7. Memory: ROM-1 Kbyte Mask ROM, containing bootstrap and initialization routines; SRAM-16 Kbyte, used for program and data space 8. External Memory: Provision for powering and reading from and writing to Atmel SEEPROM for operating system and initial parameter storage; Provision for None, 256 or 512 Kbytes external SRAM 9. Analog to Digital Converter: 12 bit, 4 channel signal multiplexer, 3 differential, 1 single-ended input signals, Vcc measurement-warm-up in 1 mS, Conversion time: <50 clocks (successive approximation), Programmable range and offset, External VRH and VRL, Separate VDD connection 10. Synchronous Serial Interfaces (2)-Clock and data in, clock and data out, handshake in and out 11. Piezo Buzzer control-7 bit tone register, bipolar or monopolar drive, 35568 Hz base block, tone is clock divided by 7 bit value in register, 8′″ bit is on/off control 12. Interrupt Control-3 external interrupt request lines, high true 13. Invalid address detection non-maskable interrupt 14. External I/O Device select, low true 15. RF Telemetry: QFAST Modulation method with demodulator and RF mixer circuitry, Power control for external RF Circuitry, Antenna tuning control: 4 bits, Device ID registers: 24 bit, Timing Control for automatic receive, with clock pulse stealer circuitry for Time base adjustment, Data rate 512 bits per second to 8192 bits per second 16. Wakeup Timers Timer 1-10 bit up-counter, 1 Hz drive, HIRQ on compare to value, then reset and up count again, range of programmable values is 3 sec to 1026 seconds; Timer 2-12 bit up-counter, 8 Hz drive, HIRQ on compare to value, then reset and up count again; Timer 3-12 bit up-counter, 1024 Hz drive, HIRQ on compare to value, then reset and count again 17. One-Minute Counter-modulo 60 counter driven by 1 Hz and HIRQ generator 18. Time of Day Registers 19. Watchdog monitor-Wakeup timer 1 interrupt signal is monitored and if two successive HIRQ3 signals are detected without proper watchdog supervision by the main processor then a system reset is asserted. It is to be understood that the above parameters are merely exemplary and are in no way limiting as to the scope of the present invention.

Further exemplary parameters of the present invention may include, but are not limited to, LCD Clock-clock line for external LCD display (to be used in HHP) 21. Test pins for system control bus visibility and debug 22. General purpose I/O used for pump control, but useful for other functions 23. Power On Clear Reset Circuitry The RF telemetry circuit 29 utilized within the CD 20, in one preferred embodiment, is based on QFAST technology. QFAST stands for “Quadrature Fast Acquisition Spread Spectrum Technique”, and represents a known and viable approach for modulating and demodulating data. The QFAST RF telemetry method is further disclosed in U.S. Pat. No. 5,559,828, incorporated herein by reference. The QFAST methodology utilizes an I/Q modulation and demodulation scheme that synchronously encodes clock and data onto a carrier signal of a suitable frequency, e.g., 262 KHz. The RF receive mixer and demodulator sections are implemented almost entirely on the Processor IC with only external receive amplifier circuitry and an antenna required to supplement the circuit. A method of tuning the antenna due to center frequency shifts upon laser welding the enclosure around the processor hybrid may be implemented under software control. Pre-weld tuning may be accomplished by the use of binary capacitors (capacitor chip arrays which are wire bonded during fabrication and tuned by testing and creating wire bonds as needed). The RF carrier is derived from the processor system clock. In one embodiment, the system clock operates at 1.000 MHz. Other frequency ranges may be used, as needed. The data rate is adjustable by register control over a suitable range, e.g., from 512 to 4096 bits per second, and the range of the link at 4 kb/s (kilobits/second).

The hand held programmer (HHP) 50 may be used by the patient to control the operation of the cranial device. The HHP functions as a small pager-like device which is designed to control the CD. The HHP, uses a 16 bit microprocessor as its main controller. This microprocessor may be the same as the microprocessor 21, used within the CD 20, and thus has all of the benefits and features described previously. The following is a list of optional features of the HHP 50. It is to be understood that these parameters are merely exemplary and are not limiting with respect to the scope of the present invention: (a). Package-central electronics volume is sealed against moisture ingress. (b) Battery compartment is moisture resistant. ESD protection-Internal surfaces treated for ESD protection. Size-3.5″L×2.6″W×0.65″T; (c) ICON area-above pixel area-time of day, month, date, activity icon, battery warning, alarm warning, reservoir volume (battery charge); (d) Interface-SPI, IIC or 8 bit parallel-SPI implemented to SSI of ASIC; (e) Programming-bit mapped graphics instruction set; Contrast-hardware and software command; (f) Power Consumption <20 uA ICON, <500 uA pixel area on.; (g) Any key on the keyboard can cause interrupt request, maskable; Seal/environmental-sealed to prevent moisture ingress, ESD shielded and debounced; Reset-Hardware reset; (h). Vibrator-A pager type vibrator motor is available for non-audible alerts to the user, and. can be powered down, as can UART. IrDA port receive line can be powered independently to see if external device needs attention even when UART is off. Batteries and up-conversion-Main Battery: lithium primary; Expected Battery Life—at least 2 months at average current of 1 mA. The HHP 50 may optionally be designed to support multiple languages through the use of its graphics LCD and to display continuously basic status information about the implanted device and its own operation. The HHP 50 can optionally perform RF telemetry to the cranial device at the specifications mentioned above, as well as communicate over an IrDA 1.2 compatible infrared cable-less data link at 115 Kbaud over a 30 cm range or using RF link through a Bluetooth conectivity. This range can be extended with the use of a commercially available IrDA 1.2 compliant serial port 8 foot expander which plugs into the 9 pin Sub-D connector found on personal computers and terminates with an IrDA transceiver. The HHP 50, in one embodiment, utilizes a label and membrane keypad to adapt to systems applications. Software applicable to brain stimulation is also used. The HHP 50 represents a general-purpose 8086-based product platform. Such platform is extremely flexible, yet meets the needs of small weight and size, rugged environmental protections and ease of use for the brain stimulation application.

The PPS 60 may be used by the physician or clinician to fit the cranial device 20 and electrodes 32 to the patient, and to record and document all stimulation settings (patient specific tuning). The PPS 60 communicates to the HHP 50 using an InfraRed wireless link 46, a standard in the computer industry. The HHP 50 communicates to the cranial device 20 over an RF link 44. Secure communications without error are provided by utilizing a 24 bit identification code for all components in the system along with error detection codes embedded in all data packets submitted by any device in the system.

Cranial Device Pulse Generator Performance-Stimulation Capability may include the following list of features: (a) At least three electrodes and case ground, individually controlled: biphasic pulse current, frequency, pulse width, channel assignment, monopolar or multipolar operation. (b) Up to 4 Channels: channel=common frequency and pulse duration for channel assigned electrodes (electrodes can operate in up to four channels). (c) Amplitude: each electrode: 0-12 mA cathodic or anodic current in discrete steps, e.g., steps of 0.1 mA. Simultaneous output: 20 mA (distributed). (d) Pulse Width: 25 ps (microseconds) to 1 ms (millisecond), in 10 us steps (equal for electrodes on a channel). (e) Rate: 2 ranges including normal, 0-150 pps per channel in approximately 1 pps steps, and high rate (1 channel) 160-1200 in approximately 10 pps steps. (f) Channel Timing: channel rates are regulated to prevent overlap with a method that is transparent to the patient. (g) Anode Control: 3 modes-monopolar case (any electrode (s) (−) to case), passive anodes (electrodes connected to ground), and active anode with individual amplitude control. (h) Charge Balance: assured through capacitor interface between electrode and output circuitry. (i) Soft Start: from 1 to 10 seconds, in 1 second steps. 1(j) Run Schedule: all channels of the implant turn on and off to the last stimulation settings at preset programmed times. (k) Impedance: monopolar at 4 mA: 500 Ohms typical. It is to be understood that the above list describes specific parameters or capabilities of cranial chip 20 that can be adjusted for particular desired pulse generation scenarios and applications.

Exemplary telemetry characteristics of the Cranial Device Pulse Generator Performance-Telemetry Output may include, but are not limited to (a) Battery Capacity: automatic telemetry data retrieval initiated by external programmer communication. (b) Electrode Impedance: automatic telemetry data retrieval initiated by external programmer communication. (c) Confirmations: programmable parameter changes from external equipment confirmed with back telemetry. (d) Programmed Settings: automatic telemetry data retrieval of all programmable settings initiated by external programmer communication.

Exemplary Cranial Device Pulse Generator Performance-Connector characteristics may include, but are not limited to: (a) five electrode leads with up to 4 total electrical contacts for a removable lead system with strong, reliable electrical performance (low current spread) under implanted conditions. (b) Although the connection is typically made only once for any device, the connector mechanism is designed to withstand a minimum of 10 connections. (c) The lead connector system utilizes a simple method to secure the electrode leadwire without the use of a tool.

Servicing and Diagnostic System (SDS) Features may include, but are not limited to: (a) Intuitive user interface; (b) Back-lighted flat panel screen; (c) Hidden physician screen; (d) 2-3 foot RF range; (e) Implant battery monitor; (f) Run time scheduler; (g) 4 program storage; (h) Infrared or Bluetooth communication link to clinician's programming system.

The Recharging System (RCS) features: The battery 27, e.g., a thin film lithium-ion battery, powers operation of the cranial device 20 and may be rechargeable. A charger coil 19 provides a means for coupling energy into the battery for recharging. The charger coil may optionally be located in a hat (worn by the patient). Battery charger and protection circuits 28 receive the power for recharging the battery through the charger coil 19; regulate and distribute power to the rest of the cranial device 20, as required, and monitor the status of the battery 27.

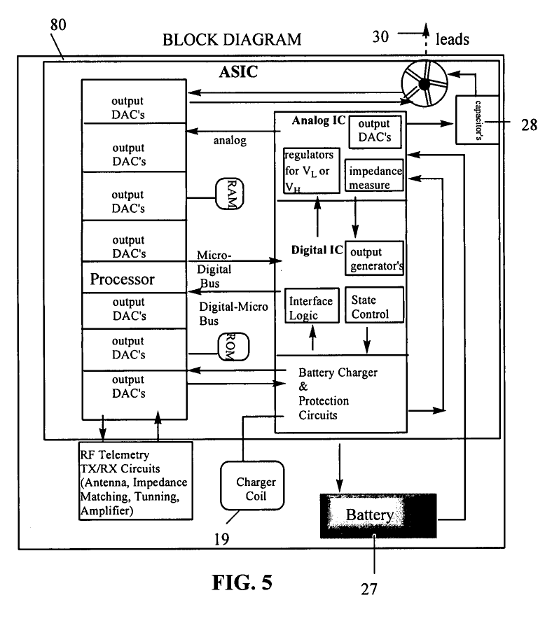

A block diagram of the circuit of cranial device 20 is shown in FIG. 5. The primary component is the application specific integrated circuit (ASIC) 80 which has the necessary SRAM and SEEROM memory, the SEEROM memory provide storage for data and control signals associated with the operation of the processor 21. (FIG. 4) The processor 21 controls digital IC 26 and directs it to generate appropriate stimulation currents for delivery through the leads 30 and electrodes 90 at the end of the leads. The digital IC 26, in turn, controls analog IC 25 so as to generate the stimulus currents. Connection with the lead (s) 30, is made through a capacitor array, so that all electrodes are capacitor coupled. A header connector 22 facilitates detachable connection of the lead (s) 30 with the cranial device 20.

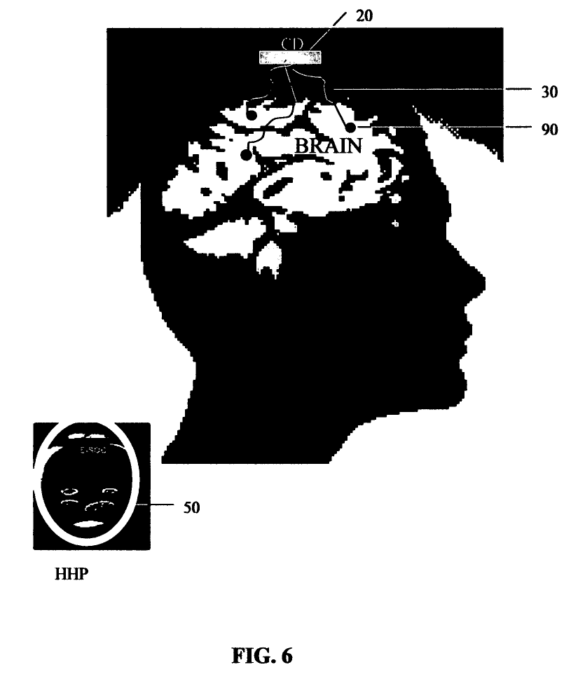

FIG. 6 represents an imaging section of the head after the cranial device and electrodes are implanted.

FIG. 7 shows the concept of using a single cranial device placed on the skull (under the scalp) and connected to multiple electrode leads. Each electrode lead reaches its target in the brain through a mini-burr hole instead of single central burr hole. In preferred embodiments, some or all of the electrode leads can be simultaneously turned on or off. Most preferably such leads are individually controllable by the control circuitry in the cranial chip device.

FIG. 8 shows the concept of using two cranial devices, one for each side of the brain, with the cranial devices placed on the skull (under the scalp) and connected to multiple electrode leads that can be simultaneously turned on and individually modulated.

It is an advantage of the invention that it can be implanted easily and effectively by the broad community of neurosurgeons rather than those just working in the specialized centers.

It is preferably possible to implant the cranial device with no general anaestesia to shorten the operating room time and the placement of the electrodes and this can be done with anatomical and or functional imaging.

It is an advantage of the invention to have the device communicate with the handheld controller that can be manipulated by the patient or the neurosurgeon/neurologist. This communication could be, but is not, limited to a RF link.

It is a feature of the invention that each of the electrodes can be individually controlled with the hand-held programmer for modulating both current and voltage and pulse profile.

It is a feature of the invention that each electrode could be switched between a “therapeutic” or “diagnostic” mode. For diagnostic purposes the electrode would sense electrical signals from a brain region and compare it to signals from a second electrode to differentiate noise levels. A real signal would be detected by the cranial chip device. The signal would be transmitted to the hand-held programmer (HHP) 60 which has a brain signal receiving processor. The said signal would then be processed and transmitted by the HHP 60 to an external or internal prosthetic device or a external or internal interface such as a computer or processor.

It is a feature of the invention that the implanted storage device can be recharged using many forms of energy including but not limited to the following energy sources: RF coupling, light such as near infra red (NIR) light, body heat, vibrational, sound or ultrasound.

### EXAMPLES

The present invention is further illustrated by the following examples which should not be construed as limiting the scope or content of the invention in any way.

### Example 1

The implantation of the cranial device is done without need for general anaesthesia. The scalp is incised a burr hole is created in the skull using standard neurosurgical techniques, all electrodes are inserted to the desired brain regions through the burr hole using currently used microelectrode recording techniques or functional imaging. Then, the cranial chip device itself would act as the burr hole cap. Once the device has been implanted and anchored to the skull, the scalp is put back and the device is ready for testing and the functioning of individual stimulus electrodes.

### Example 2

The implanted device is controlled in the clinic using the hand held controller and clinicians programming system. The electrodes are put in a therapeutic mode. The electrodes are individually adjusted to produce optimum therapeutic benefit which is determined both based on the therapeutic efficacy and by functional imaging where possible.

### Example 3

The patient has some control on level of stimulation for active electrode leads. The patient uses the hand-held controller to modulate the level of the electrode stimulation and to determine the remaining charge on the storage device. If the charge indicator falls below a certain level the patient uses the RCS and wears a hat to recharge the device. The recharge would occur in a few hours.

What is claimed:

1. A brain stimulation device, comprising:
a cranial chip that is configured to be surgically implanted between a patient's scalp and skull;
at least three stimulation leads connected to the cranial chip, wherein each lead has a plurality of stimulation electrodes thereon;
control circuitry in the cranial chip for controlling the operation of the stimulation leads and stimulation electrodes; and
a power source in the cranial chip for powering the simulation leads and the stimulation electrodes and the control circuitry.

2. The device of claim 1, wherein the cranial chip further comprises:
radio-frequency harvesting circuitry for powering the cranial chip.

3. The device of claim 1, wherein the cranial chip further comprises:
a battery for powering the cranial chip.

4. The device of claim 1, wherein the device is configured to simultaneously stimulate a plurality of locations in the patient's brain.

5. The device of claim 1, wherein the stimulation leads are individually controllable by the control circuitry.

6. The device of claim 1, wherein the stimulation electrodes on the stimulation leads are individually controllable by the control circuitry.

7. The device of claim 1, further comprising:
a wireless hand-held programmer for controlling the operation of the cranial chip.

8. The device of claim 7, further comprising:
a physician programming device in communication with the wireless hand-held programmer.

9. The system of claim 1, wherein the control circuitry generates brain stimulus pulses in accordance with a computer program stored therein.

10. The system of claim 1, wherein the stimulation electrodes are configured to simultaneously stimulate multiple targets on the same side of a patient's brain.

11. The system of claim 1, wherein the stimulation electrodes are operable in both therapeutic and diagnostic modes.

12. The system of claim 1, wherein the stimulation electrodes are positionable to target multiple locations in the brain to benefit each point in a neurocircuit.

13. The system of claim 1, wherein the control circuitry controls voltage and current characteristics of the stimulation electrodes.

14. The system of claim 7, wherein the wireless hand-held programmer controls the voltage of the stimulation electrode within 1 volt.

15. The system of claim 7, wherein the wireless hand-held programmer controls pulse duration within a change of +/−90 microseconds.

16. The system of claim 1, wherein at least a pair of the brain stimulation devices are used to each target opposites sides of a patient's brain.

17. The system of claim 16, wherein multiple mini-burr holes are used for insertion of the stimulation leads.

18. The system of claim 3, further comprising:
an inductive coupling battery charging system for charging the battery.

19. The system of claim 3, further comprising
a battery recharging system using light, heat or vibrational energy for charging the battery.

20. The system of claim 1, wherein the control circuitry comprises:
control circuits and memory circuits that cause stimulation pulses to be applied through at least one of a plurality of channels to the stimulation electrodes in accordance with a program stored within the memory circuits of the cranial chip device.

21. The system of claim 1, further comprising:
a servicing and diagnostic system for coupling with the cranial chip through an RF link or infra-red link.

22. The system of claim 1, wherein each stimulation electrode lead is dimensioned to be inserted through the burr hole in the skull.

23. The system of claim 1, further comprising:
a physician's programmer coupled to a hand-held programmer through an infra-red link or RF link to couple the physician's programmer with the cranial chip.

24. The system of claim 1, wherein the device is configured for the treatment of brain disorders.
